(19) United States
(12) Patent Application Publication
(10) Pub. No.: US 2025/0346568 A1
(43) Pub. Date: Nov. 13, 2025

(54) PRODUCING 2,5-FURANDICARBOXYLIC ACID IN THE PRESENCE OF MODIFYING ACID

(71) Applicant: Furanix Technologies B.V., Amsterdam (NL)

(72) Inventors: Jeffrey John Kolstad, Amsterdam (NL); Jan Hendrik Blank, Amsterdam (NL); Gerardus Johannes Maria Gruter, Amsterdam (NL)

(21) Appl. No.: 18/871,532

(22) PCT Filed: Jun. 13, 2023
(86) PCT No.: PCT/EP2023/065710

§ 371 Date: Dec. 4, 2024

(30) Foreign Application Priority Data
Jun. 14, 2022 (EP) 22178816.9

(51) Int. Cl. C07D 307/68; B01J 27/128

(52) U.S. Cl. CPC C07D 307/68; B01J 27/128

(57) ABSTRACT

Process for producing 2,5-furandicarboxylic acid comprising the steps of: a) oxidizing feed comprising 5-methylfurfural using an oxidizing gas in an oxidation reactor at a temperature in the range of 150 to 210° C. to obtain a crude carboxylic acid composition comprising 2,5-furamndicarboxylic acid in the presence of acetic acid and a modifying acid selected from the group consisting of carboxylic acids having a pKa of less than 3.2, and a catalyst system comprising cobalt, manganese and bromine; and b) separating solid 2,5-furamndicarboxylic acid from the crude carboxylic acid composition to obtain crude solid 2,5-furandicarboxylic acid wherein the modifying acid is added to the oxidation reactor and is selected from the group consisting of bromoacetic acid, dibromoacetic acid, 5-bromo-2-furoic acid, fumaric acid, acetoxy-acetic acid, maleic acid and furoic acid.

### TECHNICAL FIELD

The present invention relates to a process for producing 2,5-furandicarboxylic acid, specifically a process for producing 2,5-furandicarboxylic acid using 5-methylfurfural as starting material.

### BACKGROUND

2,5-Furandicarboxylic acid (FDCA) is known in the art to be a highly promising building block for replacing petroleum-based monomers in the production of high performance polymers. In recent years, 2,5-furandicarboxylic acid and the novel plant-based polyester polyethylenefuranoate (PEF), a completely recyclable plastic with superior performance properties compared to today's widely used petroleum-based plastics, have attracted a lot of attention. These materials could provide a significant contribution to reducing the dependence on petroleum-based polymers and plastics, while at the same time allowing for a more sustainable management of global resources. Comprehensive research was conducted in the field to arrive at a technology for producing 2,5-furandicarboxylic acid and PEF in a commercially viable way.

2,5-Furandicarboxylic acid is typically obtained by oxidation of molecules having furan moieties, e.g. 5-hydroxymethylfurfural (5-HMF) as well as the corresponding esters and ethers, e.g. 5-alkoxymethylfurfural, and similar starting materials, that are typically obtained from plant-based sugars, e.g. by sugar dehydration. A broad variety of oxidation processes is known from the prior art such as enzymatic and metal catalysed processes, either heterogeneous or homogeneous. WO 2014/014981, WO 2012/161968 and WO 2011/043661 describe processes using catalyst systems comprising cobalt, manganese and bromine to oxidize compounds having a furan moiety to 2,5-furandicarboxylic acid using oxygen or air as an oxidizing agent.

The purity of crude 2,5-furandicarboxylic acid product is oftentimes not sufficient for use in the manufacture of polymers having desirable properties. The product obtained by oxidation can be colored. Color is disadvantageous in that it indicates the presence of impurities. In order to objectively determine color, absorbance of a solution is to be measured at a particular wavelength such as 400 nm. Impurities which lead to color are often difficult to specifically identify making their removal difficult.

Processes have been developed for further purifying crude oxidation products. Exemplary purification processes are disclosed in WO 2014/014981 and WO 2016/195499.

It can be advantageous to be able to use 5-methylfurfural (5-MF) for oxidation into 2,5-furandicarboxylic acid as the availability of 5-methylfurfural may increase in the future. Processes which produce 5-hydroxymethylfurfural (5-HMF), as well as corresponding esters or ethers, frequently use sugar as feedstock such as glucose or especially fructose. In contrast, processes to make 5-methylfurfural can start from biomass or cellulosic materials which do not compete with the food chain and which may have reduced environmental impact. Furthermore, it could be expected that less by-products are formed in the conversion of 5-methylfurfural due to methyl group which tends to be less reactive.

While oxidation of compounds such as 5-hydroxymethylfurfural and ethers thereof has been extensively studied, less is known about oxidation of 5-methylfurfural. Soviet Union Inventor's Certificate 441877 describes the conversion of 5-methylfurfural into 2,5-furandicarboxylic acid at low yield and unknown purity. WO2011043661 mentions 5-methylfurfural as a possible feed for oxidation. Examples 3a and 3b using 5-methylfurfural gave a lower yield of 2,5-furandicarboxylic acid than Examples 1d and 1h obtained from 5-hydroxymethylfurfural, both at 100% conversion. Therefore, the product obtained from 5-methylfurfural contained more compounds other than the free diacid, i.e. 2,5-furandicarboxylic acid, than the product obtained from 5-hydroxymethylfurfural.

U.S. Pat. No. 9,321,744 describes preparing 2,5-furandicarboxylic acid by oxidizing a first and a second furan compound. It is described that an overly low ratio of the first compound cannot efficiently enhance the FDCA selectivity and the FDCA yield. The reactants of Example 3 were 1.5 g of 5-(acetoxymethyl)-2-furoic acid and 3 g of 5-methylfurfural. After introducing air at 130° C. for 1 hour and heating the mixture to 150° C. for 3 hours, the conversion rate of the 5-(acetoxymethyl)-2-furoic acid was 99.2% and of the 5-methylfurfural was 100%.

The article “Production of 2,5-furandicarboxylic acid by optimization of oxidation of 5-methyl furfural over homogeneous Co/Mn/Br catalysts” by Heng Ban et al. describes side reactions of the oxidation of 5-methylfurfural. By-products were described to consist of maleic acid, fumaric acid, trace 2-furoic acid and a little bromide derivative. There is no information on the effect of such by-products during the oxidation,

In addition, it was also found that crude 2,5-furandicarboxylic acid obtained from 5-methylfurfural can suffer from incorporation of catalyst metals, more specifically manganese and/or cobalt, into the product cake. This not only contaminates the product but also withdraws valuable catalyst components from the system that could otherwise be reused or recycled.

### DISCLOSURE OF THE INVENTION

It was an objective to improve the purity of the crude carboxylic acid obtained by oxidation of 5-methylfurfural. A further objective was to improve, i.e. reduce, the absorbance of the crude carboxylic acid obtained. Furthermore, there was a desire for a process for producing 2,5-furandicarboxylic acid from 5-methylfurfural that reduces the problem of incorporation of metals into the crude solid 2,5-furandicarboxylic acid product.

It was found that crude 2,5-furandicarboxylic acid which was obtained by oxidation of 5-methylfurfural contained a surprising impurity namely biscarbonylfuroic acid (also referred to as 5,5′-carbonyl-bis-furan-2-carboxylic acid, “BCFCA”). The presence of BCFCA during polymerization of 2,5-furandicarboxylic acid and ethylene glycol was found to lead to an undesirable increase of absorbance at 400 nm of the poly(ethylene 2,5-furandicarboxylate) produced.

Surprisingly, it now has been found that the amount of BCFCA in the crude 2,5-furandicarboxylic acid obtained by oxidation of 5-methylfurfural can be reduced by the presence of a modifying acid during oxidation.

The invention relates to a process for producing 2,5-furandicarboxylic acid comprising the steps of: a) oxidizing feed comprising 5-methylfurfural using an oxidizing gas in an oxidation reactor at a temperature in the range of 150 to 210° C. to obtain a crude carboxylic acid composition comprising 2,5-furandicarboxylic acid in the presence of acetic acid and a modifying acid selected from the group consisting of carboxylic acids having a pKa of less than 3.2, and a catalyst system comprising cobalt, manganese and bromine; and b) separating solid 2,5-furandicarboxylic acid from the crude carboxylic acid composition to obtain crude solid 2,5-furandicarboxylic acid wherein the modifying acid is added to the oxidation reactor and is selected from the group consisting of bromoacetic acid, dibromoacetic acid, 5-bromo-2-furoic acid, fumaric acid, acetoxy-acetic acid, maleic acid and furoic acid.

The pKa of the modifying acid is measured in water and is available in handbooks for many acids.

### MODES FOR CARRYING OUT THE INVENTION

The process is aimed at producing 2,5-furandicarboxylic acid. The final product obtained tend to contain further compounds besides 2,5-furandicarboxylic acid especially if the final product obtained is the crude solid 2,5-furandicarboxylic acid produced in step b). The product obtained after the washing of step c) or the hydrogenation steps d)-f), which are discussed later, tends to contain a lower amount of further compounds besides 2,5-furandicarboxylic acid.

Step a) comprises oxidation of a feed comprising 5-methylfurfural. The feed comprises at least 5-methylfurfural optionally in combination with further compounds. The feed preferably comprises at least 80% by weight of 5-methylfurfural, more preferably at least 90% by weight of 5-methylfurfural. The feed preferably consists of 5-methylfurfural. Besides the feed, the reaction mixture present in step a) comprises oxidizing gas, acetic acid, modifying acid and a catalyst system. After the reaction has started, the mixture present in step a) tends to further contain water produced by oxidation of 5-methylfurfural.

The term modifying acid is arbitrarily chosen to clearly denote the group of specific acids that were found suitable in the present process. Some suitable carboxylic acid may be formed in the conversion of 5-methylfurfural to 2,5-furandicarboxylic acid. However, this amount tends to be limited. Preferably, the modifying carboxylic acid is added to the process.

Preferably, the amount of modifying acid added is of from 0.5 to 6% by weight based on weight amount of acetic acid present in step a), preferably of from 1 to 5% by weight.

The crude solid 2,5-furandicarboxylic acid preferably contains at most 5000 parts per million by weight (ppmw) of biscarbonylfuroic acid, based on weight amount of dry 2,5-furandicarboxylic acid, more preferably at most 2000 ppmw, more preferably at most 1000 ppmw, most preferably at most 500 ppmw.

While the modifying agent can be selected from a wide range of compounds, it will be clear to the person skilled in the art that care should be taken to ensure that the oxidation reaction continues as desired besides the additional presence of the modifying acid. The modifying acid preferably is selected from the group consisting of mono- and dicarboxylic acids having from 2 to 5 carbon atoms. The modifying acid preferably is selected from the group of specific compounds consisting of bromoacetic acid, dibromoacetic acid, 5-bromo-2-furoic acid, methyl ester of 2,5-furandicarboxylic acid, fumaric acid, acetoxy-acetic acid, maleic acid and furoic acid.

More preferably, the modifying acid is selected from the group consisting of monocarboxylic acids having from 2 to 5 carbon atoms. The modifying agent is more preferably selected from the group consisting of the specific compounds bromoacetic acid, dibromoacetic acid, acetoxy-acetic acid, methyl ester of 2,5-furandicarboxylic acid and 5-bromo-2-furoic acid.

Step a) preferably is carried out at a temperature in the range of 150 to 210° C., preferably a temperature of 160 to 190° C., more preferably a temperature in the range of from 165 to 180° C. Preferably, the pressure in step a) is in the range of 700 to 2000 kPa. These parameters were found to produce 2,5-furandicarboxylic acid of good purity in good yields while at the same time enabling the reactors to be run such that the substantial heat generated by oxidation is removed by vaporization of a portion of the solvent. This is known in the art as adiabatic operation.

The catalyst system comprises cobalt, manganese and bromine either as the element or as a derivative thereof. The catalyst system preferably has a weight ratio of cobalt to manganese in the catalyst system of 10 or higher, preferably 15 or higher, and/or a weight ratio of bromine to the combined weight of cobalt and manganese in the catalyst system of 1 or higher, preferably 1.5 or higher, most preferably 2 or higher, wherein the value is preferably less than 4.0, more preferably less than 3.5. If the catalyst system comprises other metals besides cobalt and manganese in an amount of 5% by weight or more, it is preferred that the above ratios are achieved for the weight ratio of bromine to the combined weight of all metals in the catalyst system. The metals preferably are added as salts which are soluble in the reaction mixture. Typically, the amount of cobalt is selected in the range of 500 to 6000 ppm by weight, based on the weight of the feed, acetic acid and catalyst system. The amount of manganese typically is in the range from 20 to 6000 ppm by weight, based on the weight of the feed, acetic acid and catalyst system Typically, the bromine concentration would be from 30 to 8000, preferably 50 to 4500 ppm by weight of bromine, based on weight of the feed, acetic acid and catalyst system. Alternatively, the bromine content is from 3000 to 8000 ppm by weight.

The oxidizing gas can be any gas known to be suitable by the person skilled in the art. Preferably, the oxidizing gas comprises molecular oxygen. Most preferably, the oxidizing gas is air.

The reactor for carrying out the oxidation can be any typical oxidation reactor that is known in the art.

A post-oxidation step has been found to be preferred especially when employed at high temperature. Most preferred is a process wherein a post oxidation step a1) is applied after step a) at a temperature of at a temperature in the range of 150 to 210° C., more specifically of 160 to 210° C.

In step b) solid 2,5-furandicarboxylic acid is separated. This means that solid containing 2,5-furandicarboxylic acid is separated from the crude carboxylic acid composition. Not all of the 2,5-furandicarboxylic acid generally will be removed from the crude carboxylic acid composition while generally not all of the solid which is separated will be 2,5-furandicarboxylic acid.

Preferably, at least 50% by weight with respect to the weight of the dry crude solid 2,5-furandicarboxylic acid will be 2,5-furandicarboxylic acid, more preferably at least 70% by weight, more preferably at least 80% by weight, more preferably at least 90% by weight, more preferably at least 95% by weight, more preferably at least 98% by weight. Other compounds which can be present as part of the crude solid 2,5-furandicarboxylic acid are derivatives of 2,5-furandicarboxylic acid such as methyl ester of 2,5-furandicarboxylic acid, 5-hydroxymethyl-furan-2-carboxylic acid (HMFCA), 2-carboxy-5-(formyl)furan (FFCA), 5-bromo-2-furoic acid (Br-FCA) and bis-carbonyl-furoic acid (BCFCA).

In step b) at least a portion of the solid 2,5-furandicarboxylic acid is separated, that means separated from the crude carboxylic acid composition. The separation can be carried out in any way known to the person skilled in the art such as a solid-liquid separation zone in which a solid cake and a mother liquor are generated.

In a solid-liquid separation zone, a solid crude 2,5-furandicarboxylic acid cake and a mother liquor tend to be generated by separating the solid 2,5-furandicarboxylic acid. In continuous operation, at least a portion, preferably at least 60% by weight, more preferably at least 80% by weight, of the mother liquor preferably is routed from the solid-liquid separation zone to the reactor in which the oxidation occurs, also referred to as oxidation reactor, as recycled mother liquor stream.

The process of the present invention provides good results for batch processes, wherein e.g. solid precipitate comprising crude carboxylic acid composition is taken from the batch reactor, and processed in a separation zone according to step b). Modifying acids can be added to the oxidation reactor of the running batch process. Likewise, it is possible to complete a first batch process in order to analyze the resulting crude carboxylic acid composition and to provide additional modifying acids to a subsequent batch run in case the amount of biscarbonylfuroic acid and/or metal in the crude carboxylic acid composition of the first run exceeds the desired amounts.

The process of the present invention shows its full potential in continuous or semi-continuous processes as these processes are in need for suitable controlling mechanisms that allow for a minimal invasive adjustment of the running system that is suitable to counter the problem of contaminants and/or metal incorporation into the cake. Such processes generally involve continuous or intermittent addition of oxidizable feed and withdrawal of crude carboxylic acid composition comprising 2,5-furandicarboxylic acid.

Preferred is a process wherein the amount of modifying acid is increased by adding the one or more modifying acids to the oxidation reactor from external resources. External resources include but are not limited to recycle streams such as liquid separated from the 2,5-furandicarboxylic acid product for example mother liquor.

Surprisingly, it was found that crude solid 2,5-furandicarboxylic acid produced in 5-methylfurfural oxidation had an elevated content of 5-bromo-2-furoic acid. A preferred embodiment further comprises (i) obtaining at least part of the 5-bromo-2-furoic acid from the crude solid 2,5-furandicarboxylic acid, and (ii) adding at least part of the 5-bromo-2-furoic acid obtained as modifying acid to step a). The 5-bromo-2-furoic acid can be obtained in any way known to the person skilled in the art. Preferably, 5-bromo-2-furoic acid is obtained from the crude solid 2,5-furandicarboxylic acid by washing with or reslurrying the crude solid 2,5-furandicarboxylic acid in acetic acid and/or water to obtain a solution containing 5-bromo-2-furoic acid.

Preferred is a process according to the invention, wherein the process further comprises a step c) washing the crude solid 2,5-furandicarboxylic acid with a first washing solution comprising a saturated organic acid solvent having from 2 to 6 carbon atoms, preferably acetic acid, and less than 15%, preferably less than 10%, by weight of water, based on total washing solution. Such process step can be preferred to further reduce the amount of catalyst metals in the crude solid 2,5-furandicarboxylic acid. Preferred is a process wherein the crude solid 2,5-furandicarboxylic acid obtained is further washed with a second washing solution comprising water in an amount of more than 95%, preferably more than 99%, by weight with respect to the weight of the washing solution. This process step can further reduce the amount of manganese and/or cobalt.

The solid 2,5-furandicarboxylic acid can be separated from the crude carboxylic acid composition in any way known to be suitable to a person skilled in the art. A preferred separation is by solid-liquid separation more specifically by filtering or with the help of a centrifuge, more preferably a filter, more preferably a rotary pressure filter.

Preferred is a process according to the invention, wherein the crude solid 2,5-furandicarboxylic acid comprises a combined amount of cobalt and manganese of less than 3000 parts per million by weight (ppm), preferably less than 2000 ppm, preferably less than 1000 ppm, more preferably less than 300 ppm by weight of metal with respect to the weight of the 2,5-furandicarboxylic acid in the crude solid 2,5-furandicarboxylic acid.

In a preferred embodiment, the process further comprises d) contacting washed crude solid obtained in step c) with polar solvent to obtain a solution; e) contacting the solution with hydrogen in the presence of a hydrogenation catalyst at hydrogenation conditions yielding a hydrogenated solution; and f) separating purified 2,5-furandicarboxylic acid from the hydrogenated solution, preferably separating by crystallization. Suitable process conditions are for example described in WO02016/195490. Preferred process conditions comprise contacting with hydrogen at a temperature in the range of 150 to 200° C. and a contact time with the hydrogenation catalyst in the range of 5 seconds to 15 min.

Step d) suitably comprises mixing the sold obtained in step c) with polar solvent to substantially fully dissolve the 2,5-furandicarboxylic acid and any further furan containing compounds. Preferably, the polar solvent is selected from the group consisting of water, acetic acid and mixtures thereof.

It will be clear to the person skilled in the art that preferably all solution is subjected to step e) although it is possible to use part of the solution only.

Hereinafter, the invention is described in more detail using experiments.

### Example 1

The oxidation reactor is a 600 ml stirred pressure vessel, with two impellors. The reactor is pre-charged with a mixture having a total weight of 310 grams. The mixture comprises catalyst components provided as cobalt(II) acetate tetrahydrate, manganese(II) acetate tetrahydrate, and HBr as 48% by weight (wt %) in water. The amounts of the catalyst components are such as to yield a mixture which contained 3300 ppm Co, 188 ppm Mn and 7000 ppm Br. Water is added in an amount to result in 5 wt % of the total mixture, after accounting for the water introduced as part of the catalyst components. The balance is acetic acid in the comparative experiments. The balance is acetic acid with added modifying acid in the experiments according to the invention. The amount of modifying acid is described in Table 1.

The oxidation reactor is purged, pressurized, and heated to the desired operating temperature with stirring at 2000 rpm. The feed is 5-methyl furfural (5-methylfurfural). The process is started with a typical feed rate 8.3 mmol/minute. This feed rate was continued for 60 minutes (total feed 500 mmol) in the first set of experiments (Comp. A1, Comp. A2, A3 and A4) and for 30 minutes (total feed 250 mmol) in the second set of experiments (Comp. B1 and B2). A flow rate of lean air (8% oxygen) is started at a typical flow rate of 10 normal L/minute. The reaction typically begins within 3 minutes, noticed by a sharp decrease in oxygen in the outlet and an increase in CO and CO₂. During the reaction heat is generated, and a vapor stream is taken overhead and condensed. This vapor stream comprises mainly of acetic acid and water. The amount of solvent captured in the overhead is continuously monitored, and made up in the oxidation reactor with a fresh flow of solvent to the reactor.

The typical operating pressure is 12 to 14 barg at 160° C. and 17.5 barg at 175° C.

At the end of the desired feed period, the contents of the oxidation reaction are subjected to post-oxidation. Post-oxidation was conducted by stopping the flow of lean air for 1 minute and then reestablishing lean air flow at 4 NI/min for 20 minutes while maintaining the reaction temperature at 160° C.

Solids were separated by filtration and the cake obtained was washed twice with 1 part solvent (95 acetic acid to 5 parts water, by weight) to 1 part estimated dry cake weight each time.

The color of the cake was assessed visually.

The cake absorbance was measured by mixing 300 mg of crude 2,5-furandicarboxylic acid with 10 ml of dimethyl sulfoxide (DMSO). To ensure complete dissolution, the solution was allowed to stand for 4 hours. The absorbance of this solution was measured in a 1 cm cell in a UV/VIS photospectrometer against a DMSO standard using a wavelength of 400 nm.

The amount of added modifying acid is weight amount based on weight amount of acetic acid.

The amount of cobalt and manganese present in the cake were determined by inductively coupled plasma or ICP analysis.

The amount of BCFCA was determined by ultra high performance liquid chromatography (UPLC) gradient elution on a Waters C18 column. Eluent A was water containing 0.2 vol % trifluoroacetic acid and eluent B was a 50/50 by volume mixture of methanol and acetonitrile.

The results are shown in Table 1.

TABLE 1
Operation at 160° C. for 1 hour

| | | Amount | | | | | UV |
|---|---|---|---|---|---|---|---|
| | | of feed | BCFCA | Co | Mn | Cake | absorbance |
| # | Modifying acid | (mmol) | (ppm) | (ppm) | (ppm) | color | 400 nm |
| Comp. | — | 500 | 710 | 2120 | 360 | Yellow | 0.110 |
| A1 | | | | | | | |
| Comp. | — | 500 | 738 | 2490 | 400 | Yellow | 0.259 |
| A2 | | | | | | | |
| A3 | 2.17% wt of | 500 | 40 | 7 | 0 | Light | 0.04 |
| | methyl ester of | | | | | yellow | |
| | 2,5- | | | | | | |
| | furandicarboxylic | | | | | | |
| | acid and 2.17% | | | | | | |
| | wt of | | | | | | |
| | bromoacetic | | | | | | |
| | acid | | | | | | |
| A4 | 2.13% wt of | 500 | 403 | 16 | 1 | Good to | 0.05 |
| | bromoacetic | | | | | Light | |
| | acid | | | | | Yellow | |

Table 1 shows that the addition of modifying acid allows to strongly reduce the amount of metals besides the amount of biscarbonylfuroic acid (BCFCA) present in the cake.

In the second set of experiments, a temperature of 175° C. was applied for 30 minutes at a typical feed rate of 8.3 mmol/minute (total feed 250 mmol). In comparative experiment B1, activity started visibly dropping after 29 minutes. This was followed by 15 minutes of post-oxidation at the same temperature.

The cake was separated by filtration and washed twice with 1 part solvent (95 acetic acid to 5 parts water, by weight) to 1 part estimated dry cake weight each time.

The results are shown in Table 2.

TABLE 2
Operation at 170° C. for 30 minutes

| | | Amount | | | | UV |
|---|---|---|---|---|---|---|
| | Modifying | of feed | BCFCA | Co | Mn | absorbance |
| # | acid | (mmol) | (ppm) | (ppm) | (ppm) | 400 nm |
| Comp. | — | 250 | 1959 | 3273 | 322 | 0.38 |
| B1 | | | | | | |
| B2 | 2.12% wt | 250 | 473 | 1480 | 645 | 0.04 |
| | of bromo- | | | | | |
| | acetic | | | | | |
| | acid | | | | | |

### Example 2

Ethylene glycol (MEG) and 2,5-furandicarboxylic acid (FDCA) were mixed in an MEG:FDCA molar ratio of 1.24 along with Sb₂O₃ as a catalyst, present at 240 ppm of antimony.

In Experiment C, 100 ppm of BCFCA was added to the reaction mixture. In Experiment D, 50 ppm BCFCA was added. In Experiment E, no BCFCA was added.

Each of the mixtures was subjected to esterification without addition of an esterification catalyst for a period of 165 minutes at 220° C. The pressure was reduced, and prepolycondensation was started. The pressure was further reduced and the ester composition with the catalyst was subjected to a polycondensation at a temperature of 260° C. for 60 minutes.

The cake absorbance was measured by mixing 300 mg of crude 2,5-furandicarboxylic acid with dimethyl sulfoxide (DMSO) to prepare 10 ml solution. To ensure complete dissolution, the solution was allowed to stand for 4 hours. The absorbance of this solution was measured in a 1 cm cell in a UV/VIS photospectrometer against a DMSO standard using a wavelength of 400 nm.

The number average molecular weight was determined through the use of gel permeation chromatography (GPC) performed at 25° C. Polystyrene standards were used for calculating the number average molecular weight (Mₙ). As eluent a solvent mixture of chloroform:2-chlorophenol 6:4 (vol/vol) was used.

TABLE 3
Effect of BCFCA on polymer

| | BCFCA | | |
|---|---|---|---|
| # | (ppm) | UV absorbance 400 nm | Mₙ |
| C | 100 | 0.010 | 17152 |
| D | 50 | 0.007 | 19477 |
| E | 0 | 0.004 | 18360 |

The above shows that the presence of BCFCA at polymerization of 2,5-furandicarboxylic acid and ethylene glycol can lead to increase of UV absorbance of the polymer obtained.

1. A process for producing 2,5-furandicarboxylic acid which process comprises the steps of:
a) oxidizing feed comprising 5-methylfurfural using an oxidizing gas in an oxidation reactor at a temperature in the range of 150 to 210° C. to obtain a crude carboxylic acid composition comprising 2,5-furandicarboxylic acid in the presence of acetic acid and a modifying acid selected from the group consisting of carboxylic acids having a pKa of less than 3.2, and a catalyst system comprising cobalt, manganese and bromine; and
b) separating solid 2,5-furandicarboxylic acid from the crude carboxylic acid composition to obtain crude solid 2,5-furandicarboxylic acid,
wherein the modifying acid is added to the oxidation reactor and is selected from the group consisting of bromoacetic acid, dibromoacetic acid, 5-bromo-2-furoic acid, fumaric acid, acetoxy-acetic acid, maleic acid and furoic acid.

2. The process according to claim 1, wherein the modifying acid is selected from the group consisting of mono- and dicarboxylic acids having from 2 to 5 carbon atoms.

3. The process according to claim 2, wherein the modifying acid is selected from the group consisting of bromoacetic acid, dibromoacetic acid, acetoxy-acetic acid and 5-bromo-2-furoic acid.

4. The process according to claim 1, wherein the crude solid 2,5-furandicarboxylic acid contains at most 500 parts per million by weight of 5,5′-carbonyl-bis-furan-2-carboxylic acid, based on weight amount of dry 2,5-furandicarboxylic acid.

5. The process according to claim 1, wherein the amount of modifying acid is of from 0.5 to 6% by weight based on weight amount of acetic acid present in step a), preferably of from 1 to 5% by weight.

6. The process according to claim 1, wherein the process further comprises
c) washing the crude solid 2,5-furandicarboxylic acid with a first washing solution comprising a saturated organic acid solvent having from 2 to 6 carbon atoms and less than 15% by weight of water, based on total amount of washing solution, preferably followed by washing with a second washing solution comprising water in an amount of more than 95% by weight with respect to the weight of the washing solution.

7. The process according to claim 1, wherein the crude solid 2,5-furandicarboxylic acid comprises 2,5-furandicarboxylic acid in an amount greater than 95%, preferably greater than 98%, by weight with respect to the weight of the dry crude solid 2,5-furandicarboxylic acid.

8. The process according to claim 1, wherein the crude solid 2,5-furandicarboxylic acid comprises a combined amount of cobalt and manganese of less than 1000 ppm, preferably less than 300 ppm, by weight as metal with respect to the weight of the 2,5-furandicarboxylic acid in the crude solid 2,5-furandicarboxylic acid.

9. The process according to claim 6, which process further comprises
d) mixing washed crude solid obtained in step c) with polar solvent to obtain a solution;
e) contacting the solution with hydrogen in the presence of a hydrogenation catalyst at hydrogenation conditions yielding a hydrogenated solution; and
f) separating purified 2,5-furandicarboxylic acid from the hydrogenated solution.
